(19) United States
(12) Patent Application Publication
(10) Pub. No.: US 2009/0264776 A1
(43) Pub. Date: Oct. 22, 2009

(54) MEASUREMENT OF PHYSIOLOGICAL CHARACTERISTICS

(76) Inventor: Terence Vardy, Tweed Heads (AU)

Correspondence Address: LADAS & PARRY, 5670 WILSHIRE BOULEVARD, SUITE 2100, LOS ANGELES, CA, 90036-5679, US

(21) Appl. No.: 12/364,210

(22) Filed: Feb. 2, 2009

(30) Foreign Application Priority Data
Apr. 17, 2008 (AU) 2008901911

(51) Int. Cl. A61B 5/02; A61B 5/05

(52) U.S. Cl. 600/506; 600/547

(57) ABSTRACT

A system for measuring physiological aspects has a non-invasive monitor configured to generate monitor signals relating to fluid characteristics in the body. A computational device is operatively connected to the monitor and is configured to process the monitor signals to generate characterising data relating to at least one of regional fluid volumes, intra/extracellular fluid volume ratios and blood flow. A data output device is connected to the computational device and is configured to output the characterising data. A method and a computer program product for measuring physiological characteristics are also provided.

### FIELD OF THE INVENTION

The present invention generally relates to the measurement of physiological characteristics. In particular, the present invention generally relates to a method, a system and a software product for measuring physiological characteristics of humans or animals.

### BACKGROUND ART

[Mere reference to background art herein should not be construed as an admission that such art constitutes common general knowledge in relation to the invention.]

Redistributions of fluids between segments of a body are often of central clinical importance, particularly in humans. This includes redistributions of fluids between the intra-cellular and extra-cellular compartments within the segments. Measurement of these redistributions can be useful, particularly for monitoring and assessing the response and adaptation of the body to various orthostatic and anti-orthostatic dysfunctions.

However, currently employed methods of measuring redistributions of fluids between segments of a body are either invasive or bulky and expensive. For example, tracer dilution techniques involve invasively administering a dose of an appropriate tracer to the body, collecting blood samples, and measuring the tracer. Alternatively, MRI technologies can be used but MRI equipment is both costly and bulky, making it impractical readily to measure redistributions of fluids between segments of a body. A further disadvantage of these techniques is that they do not yield easily used real-time data during physiological stress or clinical diagnosis. Nor are there auto-regulation or homoeostatic balance ranges that can be applied generally to both adults and infants. An elderly adult, mature adult, young adult, child, infant (male or female) may have different ranges of auto-regulation.

### SUMMARY OF THE INVENTION

According to a first aspect of the invention, there is provided a system for measuring physiological characteristics, the system comprising:

a non-invasive monitor configured to generate monitor signals relating to fluid characteristics in the body;

a computational device operatively connected to the monitor and configured to process the monitor signals to generate characterising data relating to at least one of regional fluid volumes, intra/extracellular fluid volume ratios and blood flow; and

a data output device connected to the computational device and configured to output the characterising data.

The redistributions of fluids between segments of a body affect cardiovascular function, water balance and perhaps skeletal muscle function through physiological mechanisms. Furthermore, redistribution of fluids, particularly lymph fluids, in various regions of the body such as the lower extremities, can have deleterious effects on the body as a whole. This invention provides a means whereby the physiological aspects of a body related to the distribution of fluids can be understood to some extent. This can be done with characterization of the fluid redistributions themselves, and thus of associated changes in cardiovascular and hemodynamic parameters.

More particularly, the present invention relates to monitoring body segment impedances for determining fluid volumes and fluid flows in a body in real time. Other physiological data such as blood pressure, temperature, ECG, the oxygen saturation level of the blood and blood flow characteristics may be incorporated for analysing trends of an auto-regulation phase particular to an individual.

This invention provides an improved method of analysing data from physiologic monitors using a computational device and a software product. In one embodiment, the computational device or computer and software product are configured for determining fluid volumes and fluid flows in a body in at least near real time which overcomes or ameliorates one or more of the disadvantages or problems described above, or which at least provides a useful alternative. One embodiment of the invention discloses a critical real time analysis combining the display of different types of data simultaneously. This results in more time efficient and effective decision making and the choice of the correct medical intervention being applied.

For the sake of convenience, the term “monitor” should be understood to mean one or more monitors.

The non-invasive monitor may be one of an impedance plethysmographic (IPG) monitor and an impedance spectroscopic (EIS) monitor.

Where applicable, the IPG monitor may be a Tetrapolar High Resolution Impedance Meter (THRIM).

The IPG and EIS monitors may typically be separate instruments. The THRIM may operate on a set frequency of 51.2 Kilohertz and may be configured to display for body segments. In one embodiment, the monitors may be a single instrument with all fluids monitored down to intracellular and extracellular fluid levels.

The system may include at least two excitation electrodes for providing an electrical stimulus across selected body areas.

The monitor may have a number of electrodes that are configured to be placed in a non-invasive manner into operative engagement with the body, such that at least one electrode engages each of a number of respective segments of the body.

The monitor may be configured to use at least 20 different frequencies. Instead, the monitor may be configured to use at least 40 different frequencies at log intervals of between 3 and 300 kHz.

The monitor may be an EIS monitor which is configured to use a constant current transformer coupled excitation stage in conjunction with a digital demodulation stage to supply both resistant and reactive impedance components, the monitor including a microprocessor system that can be coupled to the computational device with a suitable interface.

The microprocessor system may be configured to store data in the form of impedance parameters and signal waveform segments before communicating that data to the computational device. The computational device may be configured to store an executable deconvolution algorithm for processing that data to generate parameters for an R-C equivalent circuit used to generate characterising data to model intravascular, interstitial and intracellular fluid spaces.

The data output device may be configured to output the characterising data in one of the following forms:

a) real-time

b) a replay of previously recorded characterising data

c) together with mathematically reconstructed waveforms.

According to a second aspect of the invention, there is provided a method for measuring physiological characteristics, the method comprising the steps of:

engaging a non-invasive monitor with a body, the non-invasive monitor being configured to generate monitor signals relating to fluid characteristics in the body;

processing the monitor signals to generate characterising data relating to at least one of regional fluid volumes, intra/extracellular fluid volume ratios and blood flow; and

outputting the characterising data.

The step of engaging a non-invasive monitor with a body may include the step of engaging an electrode montage with the body such that five segments of the body are monitored, namely a head segment, a chest segment, a splanchnic segment, a pelvic segment and a leg segment.

The electrodes may form part of a montage of ten electrodes, two electrodes being placed into engagement with each of a head segment, a chest segment, a splanchnic segment, a pelvic segment and a leg segment.

The chest segment, the splanchnic segment, the pelvic segment and the leg segment may be monitored using a tetrapolar impedance system, while the head segment may be monitored using a bipolar impedance configuration.

The step of processing the monitor signals may include the step of processing the monitor signals to generate resistance and reactive capacitance data and to process that data to generate the characterising data.

The step of outputting the characterising data may include the step of normalising the data and displaying the data visually.

The characterising data may be output in one of the following forms:

a) real-time

b) a replay of previously recorded characterising data

c) together with mathematically reconstructed waveforms.

It follows that the system and method of the invention output and monitor fluid data, including fluid movement data, between the brain/head, chest, abdomen, pelvis, thigh, lower legs, and arms regions of a body. The output fluid information of these body regions may provide descriptions of the hemodynamic and volume responses in a human body in conjunction with other physiological data.

The output may be used to characterise regional fluid volumes, intra/extracellular fluid volume ratios, hemodynamic status and blood flow in real time during clinical and research protocols that allows the quantification of segmental blood flows, total segmental volumes, and segmental compartment volumes in real time. In the event of trauma and in the absence of outward signs of injury, the detection of abnormal blood pooling or flow is critical in the diagnosis and application of specific treatment.

The IPG and EIS monitors can further comprise two, four, or six electrodes non-invasively placed in engagement with the body. The use of an IPG or EIS may be determined by the number of electrode inputs being used. For example, 2 to 4 electrodes are used with an IPG and 6 with an EIS.

The calculated blood flow or fluid flow in each segment is typically displayed in millilitres per second, litres per hour, or cubic centimetres per minute (cc/min).

Typically the microprocessor system will be connected to the computer through Jo either the RS232 or USB serial interfaces.

The microprocessor system may store impedance parameters and signal waveform segments prior to supplying the data to the connected computer. The computer may be programmed with a software product, in accordance with the invention such that the computer uses a de-convolution algorithm on data obtained from the microprocessor system to obtain parameters for an R-C equivalent circuit used to model the intravascular, interstitial and intracellular fluid spaces.

In one embodiment, all of the EIS will be portable. Furthermore, the EIS may be configured to monitor the fluid data itself.

The EIS electrode leads may need shielding along the length of the electrode leads to prevent interference such as environmental interference. The electrodes may be disposable EKG electrodes.

The instruments of the system and method may be battery powered, power supply powered, or a combination of both. A voltage level indicator may be provided on all battery powered devices (if any) and the voltage level is read prior to conducting tests to ensure adequate battery power is available for the tests.

According to a third aspect of the invention, there is provided a computer program product comprising a computer usable medium including a computer readable program for measuring physiological aspects, wherein the computer readable program, when executed on a computer, causes the computer to:

process data received from a non-invasive monitor engaged with the body to generate characterising data relating to at least one of regional fluid volumes, intra/extracellular fluid volume ratios and blood flow; and

output the characterising data.

The computer readable program, when executed on a computer, may cause the computer to output the characterising data in one of the following forms:

a) real-time

b) a replay of previously recorded characterising data

c) together with mathematically reconstructed waveforms.

The following description is not intended to limit the scope of the above paragraphs or the scope of the claims. As such, the purpose of the following description is to describe to a person of ordinary skilled in the art how to put an embodiment of the invention into practice.

### BRIEF DESCRIPTION OF THE DRAWINGS

In order that the invention may be more readily understood and put into practice, one or more preferred embodiments thereof will now be described, by way of example only, with reference to the accompanying drawings, in which:

FIG. 1 shows a system, in accordance with the invention, for measuring physiological characteristics;

FIG. 2 shows one possible example of an electrode montage of the system, in use;

FIG. 3 shows an output of characterising data generated by the system of FIG. 1 using the electrode montage of FIG. 2;

FIG. 4 shows an output of normalized characterising data for four segments of a body on a test subject in two different cases (4a and 4b);

FIG. 5 shows another possible example of an electrode montage, of the system, in use;

FIG. 6 shows an output from an IPG monitor using the electrode montage in FIG. 5 in two different cases (sweep 1 and sweep 2);

FIG. 7 shows an output plot of various parameters over a number of sequences; and

FIG. 8 shows example output results from the EIS.

FIG. 9 shows a flow chart indicating an initial stage of a method, in accordance with the invention, carried out by a system, also in accordance with the invention, when a software product, also in accordance with the invention, is executed by components of the system.

FIG. 10 shows a flow chart indicating a log in stage of the method of FIG. 9.

FIG. 11 shows a flow chart indicating a digital control and alert settings stage, a stage for adding nodes and setting dictation characteristics, and a stage for changing sensor settings.

FIG. 12 shows a flow chart indicating a stage for capturing data.

FIG. 13 shows a flow chart indicating a stage for analysing data, a stage for carrying out a second phase analysis on the data, and a comparison phase.

FIG. 14 shows a flow chart indicating a stage for either importing or exporting data.

FIG. 15 shows a flow chart indicating a stage for either e-mailing or printing data.

FIG. 16 shows a flow chart indicating a stage for setting up hardware that executes the software product.

FIG. 17 shows a flow chart indicating a stage for exiting a software product and a stage for selecting help.

### DESCRIPTION OF PREFERRED EMBODIMENT

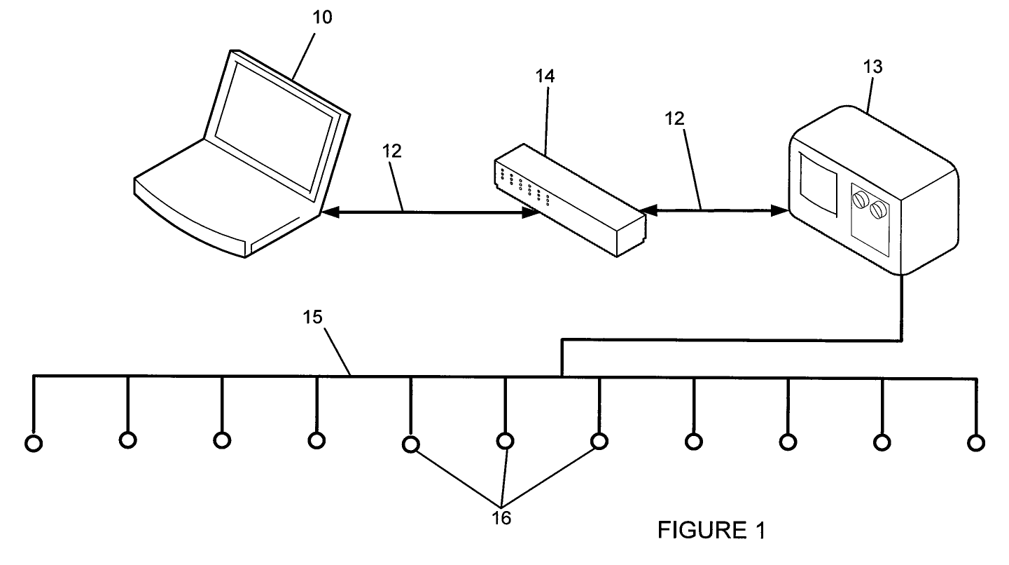

FIG. 1 shows, broadly, a system, in accordance with the invention, for measuring physiological aspects of a body. The system includes a computer 10 that defines a computational device and a data output device of the invention. Output data 11 generated by the computer 10 can be graphs displayed on a screen of the computer 10, but may take any suitable form, such as tables or metrics. The computer 10 is connected to impedance spectroscopic (EIS) and/or impedance plethysmographic (IPG) monitors represented with reference 13 in FIG. 1, via a microprocessor system 14 which is in turn connected via serial cable 12. The computer 10 can be a stationary desktop model or preferably a laptop computer for portability. For the sake of convenience, the monitors will be referred to as the monitor 13.

In the embodiment shown in FIG. 1, the cable 12 is connected to the computer 10 using an RS-232 serial interface, but it is envisaged that other interfaces could be used, such as a universal serial bus (USB) interface.

Up to 40 different frequencies, at log intervals between 3 and 300 KHz are used by the monitor 13. Where the monitor is an EIS monitor, it uses a constant current transformer coupled excitation stage in conjunction with a digital demodulation stage to supply both resistive and reactive impedance components. Thus, the monitor 13 can be configured to generate monitor signals relating to fluid characteristics in the body, in this example, said fluid characteristics being resistive and reactive impedance components. The microprocessor system 14 stores data in the form of impedance parameters and signal waveform segments carried by the monitor signals prior to communicating the data to the computer 10 for processing to generate characterising data for on-line real time analysis and display.

A software product, in accordance with the invention, when executed by the computer 10, uses a de-convolution algorithm applied to the impedance parameters and signal waveform segments to obtain parameters for an R-C equivalent circuit used to model the intravascular, interstitial, and intracellular fluid spaces.

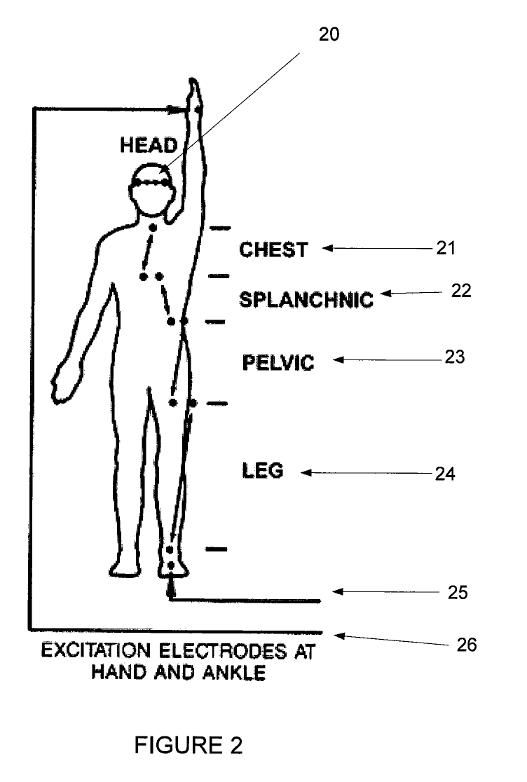

The monitor 13 is connected to a montage 15 having ten electrodes 16. As depicted in FIG. 2, a suitable electrode montage is provided which divides the body into five segments for segmental blood flows and volume change analysis. These are a head segment 20, a chest segment 21, a splanchnic segment 22, a pelvic segment 23, and a leg segment 24. The chest segment 21, splanchnic segment 22, pelvic segment 23, and leg segment 24 of the body are monitored by the monitor 13 using a tetrapolar impedance system. The head segment 20 is monitored by the monitor 13 using a bipolar impedance configuration for monitoring cerebral (head) blood flow responses.

Electrodes 25 and 26 are excitation electrodes for supplying a minute electrical charge at different frequencies to the body to be read by the other electrodes.

The electrodes need shielding along the length of the electrode leads to prevent interference such as environmental interference. A suitable electrode is a general purpose disposable EKG electrode.

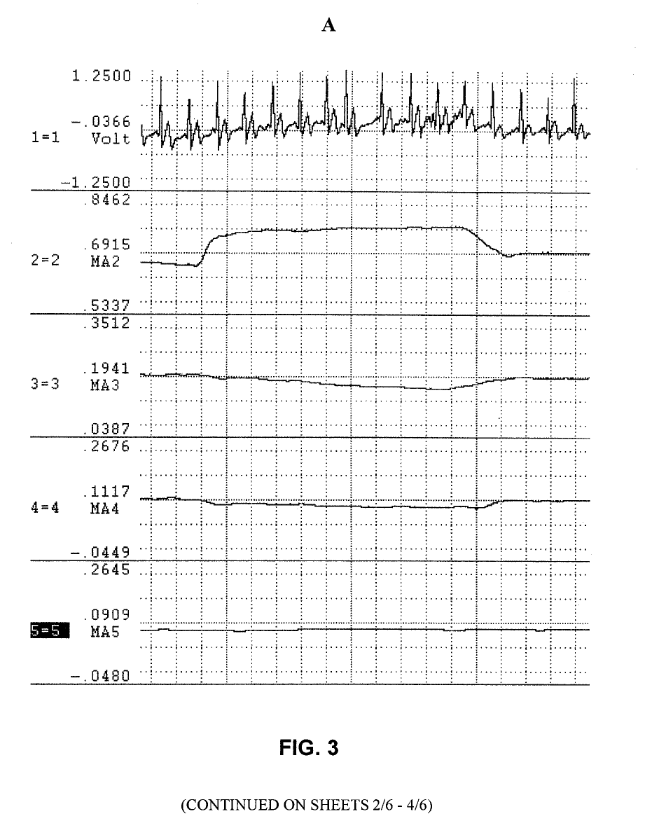

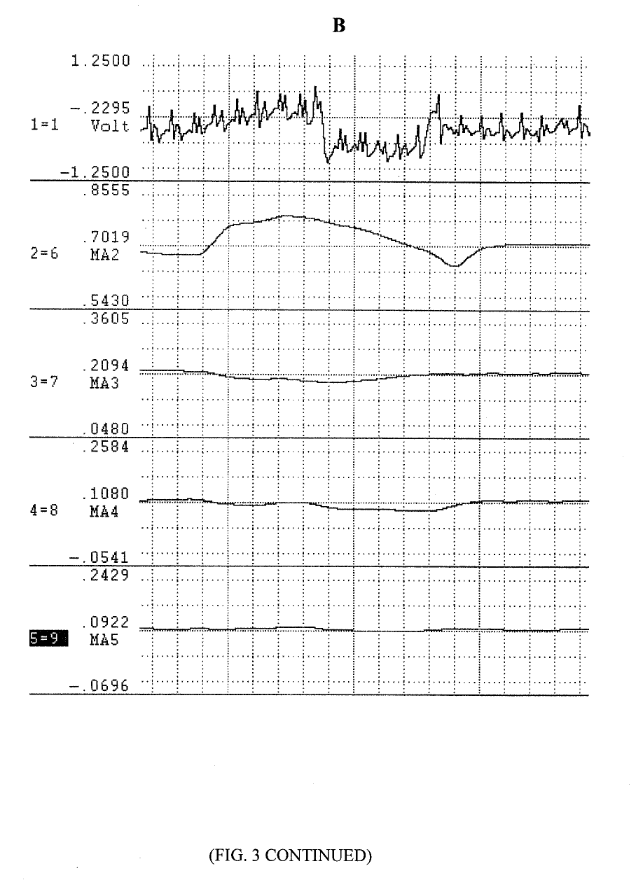

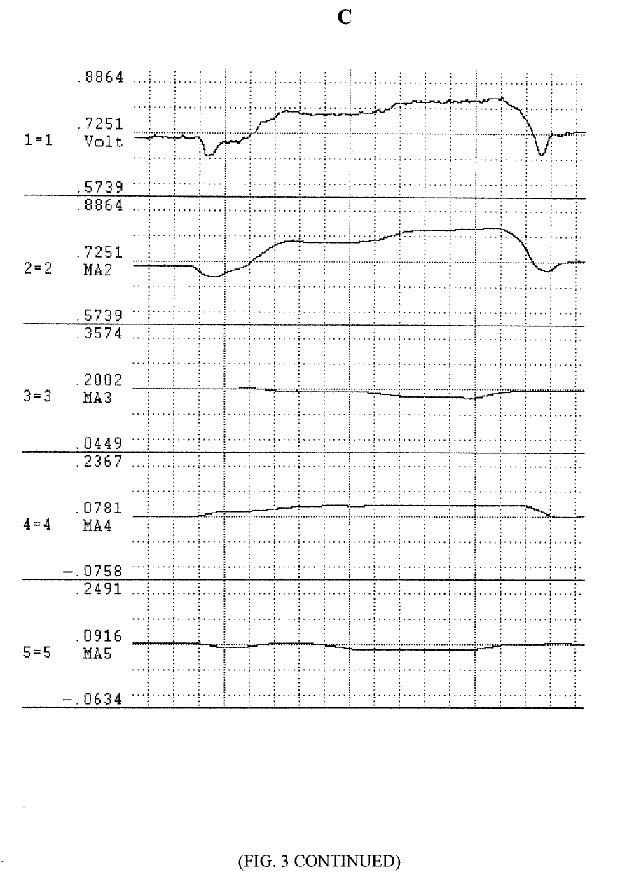

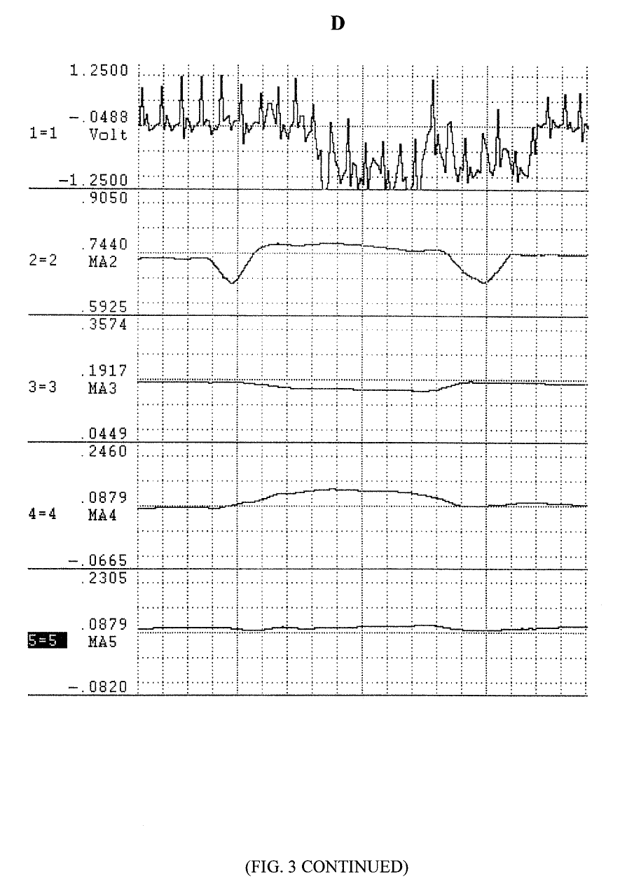

Normalised output results from the electrode montage of FIG. 2 are depicted in FIG. 3. Four readings (A to D) were taken from subjects for the five segments described above. Readings A to D are different cases/patients used and the five segments correspond to the following five body segments: head, thorax, abdomen, pelvis, and thigh.

When the computer 10 executes the software product of the invention, the computer 10 generates data relating to segmental resistance, volume, and normalized volume changes during exercise stress. These are shown in the following table for a test subject ‘TCV’. Of the three columns of data, the first column is the resistance in ohms, the second column is extracellular volume (VE) in ml, and the third column is VE over body surface area ml/m̂2.

| | | TCV | TCV | TCV |
|---|---|---|---|---|
| | | DEL R - | DEL VE - | DELVE/BSA - |
| CASE | SEGMENT | Ohms | ml | ml/M² |
| A | THIGH | 8.440 | −207.992 | −100.674 |
| | PELVIC | −3.090 | 408.426 | 197.689 |
| | SPLANCHNIC | −2.177 | 912.390 | 441.621 |
| | CHEST | 0.023 | 20.393 | 9.871 |
| B | THIGH | 5.573 | −155.331 | −75.184 |
| | PELVIC | −2.047 | 369.329 | 178.765 |
| | SPLANCHNIC | −2.463 | 734.382 | 355.461 |
| | CHEST | −0.283 | −65.893 | −31.894 |
| C | THIGH | 5.147 | −174.686 | −84.553 |
| | PELVIC | −1.527 | 218.145 | 105.588 |
| | SPLANCHNIC | 1.347 | −1334.207 | −645.792 |
| | CHEST | −0.773 | 557.791 | 269.986 |
| D | THIGH | 2.543 | 199.670 | 96.646 |
| | PELVIC | −1.607 | 119.225 | 57.708 |
| | SPLANCHNIC | 2.340 | −46.681 | −22.595 |
| | CHEST | 0.997 | −753.670 | −364.797 |

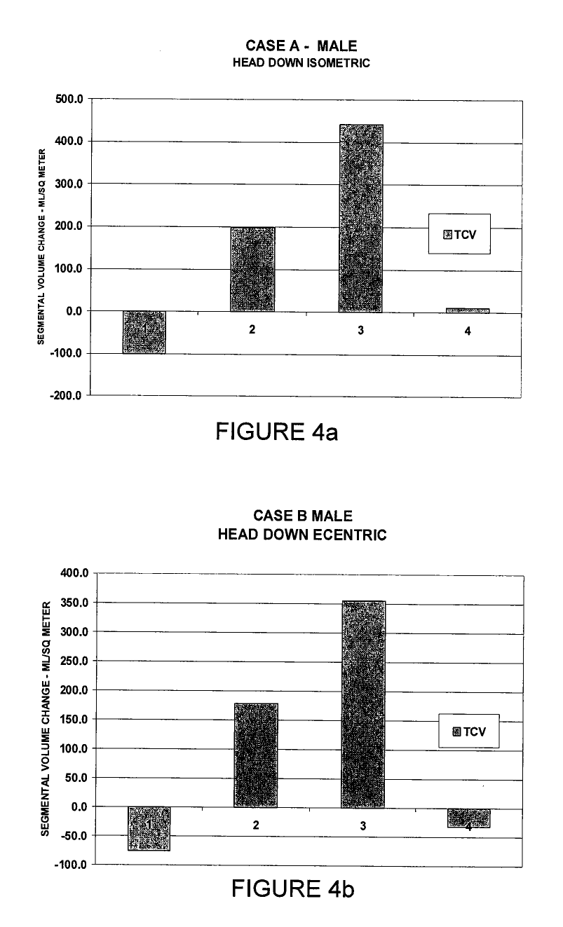

These results are shown as bar charts in FIG. 4 for two cases, namely with the subject's head down during isometric exercise and head down during eccentric exercise. The numbers 1 through to 4 correlate to the thigh, pelvic, splanchnic, and chest segments respectively. The positive result in FIG. 4a relates to when the body is upright, and the negative result in FIG. 4b to when the head of the subject is down.

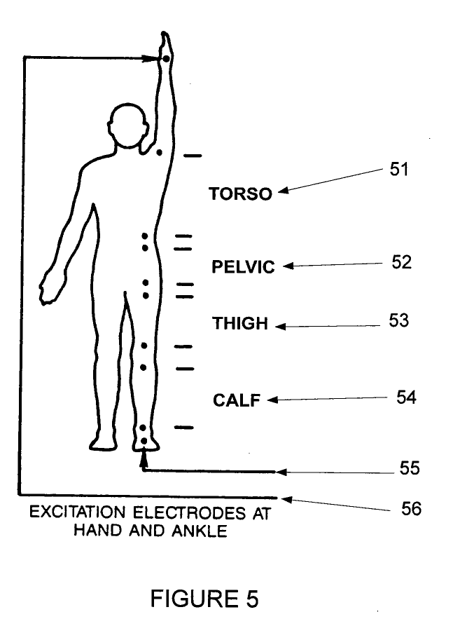

Example data obtained using IPG measurement techniques for three subjects (TBA, DLB, and HJB) using the electrode montage shown in FIG. 5 can be seen in the following table.

| | | TBA | TBA | DLB | DLB | HJB | HJB |
|---|---|---|---|---|---|---|---|
| CASE | SEGMENT | DEL VE | DELVE/BSA | DEL VE | DELVE/BSA | DEL VE | DELVE/BSA |
| A | CALF | 3.772 | 1.968 | −18.787 | −10.385 | −19.971 | −9.926 |
| | THIGH | 265.502 | 138.499 | 150.279 | 83.073 | 298.018 | 148.120 |
| | PELVIC | 255.754 | 133.413 | 113.079 | 62.509 | 400.751 | 199.181 |
| | TORSO | 794.967 | 414.694 | 158.423 | 87.575 | −216.320 | −107.515 |
| B | CALF | −153.680 | −80.167 | 30.285 | 16.741 | −89.788 | −44.626 |
| | THIGH | 349.239 | 182.180 | 1252.910 | 692.598 | 63.937 | 31.778 |
| | PELVIC | 487.859 | 254.491 | 2021.763 | 1117.614 | 108.295 | 53.824 |
| | TORSO | −990.734 | −516.815 | 1912.500 | 1057.214 | −803.512 | −399.360 |
| C | CALF | −46.212 | −24.106 | −122.378 | −67.650 | not tested | not tested |
| | THIGH | 441.211 | 230.157 | 246.071 | 136.026 | not tested | not tested |
| | PELVIC | 234.088 | 122.111 | 69.941 | 38.663 | not tested | not tested |
| | TORSO | 339.026 | 176.853 | 410.133 | 226.718 | not tested | not tested |
| D | CALF | −355.589 | −185.492 | −0.691 | −0.382 | −140.574 | −69.868 |
| | THIGH | −210.507 | −109.810 | −92.034 | −50.876 | 234.810 | 116.705 |
| | PELVIC | 198.972 | 103.793 | −86.370 | −47.745 | −391.168 | −194.418 |
| | TORSO | 385.903 | 201.306 | −312.303 | −172.638 | 60.324 | 29.982 |

Impedance data can be recorded from the body segments in, or near, real time and within a clinical environment. A vector board version can also be used to generate resistance or reactance recordings at various EIS input sampling frequencies.

An example output plot from the IPG of resistance (Ro) vs capacitive reactance (Xc) for a test subject is illustrated in FIG. 6. The plot has two sweeps; the first sweep being taken after the subject has had his leg low for 2 minutes and the second sweep being taken after the subject has had both legs put up on a desk for 4 minutes. It is clearly seen that although the capacitive reactance is very similar in each case, that the resistance has been increased by about 5 ohms resulting in a shift to the right in the plot.

Where the IPG or EIS monitor is a Tetrapolar High Resolution Impedance Meter (THRIM), such a monitor is a swept frequency THRIM that can provide the Ro and Xc data for a total of 40 log separated excitation frequencies from 3.2 to 281 KHz. The THRIM also provides a constant current, transformer isolated sine wave excitation signal, with a means being provided to trim the excitation current. The THRIM also includes 12 bit analogue to digital sampling for the Ro and Xc data as well as an RS-232 serial or USB interface, and computer software. The software product of this invention allows for configuration of the THRIM, control of the frequency sweeping function, and retrieval and storage of the impedance data.

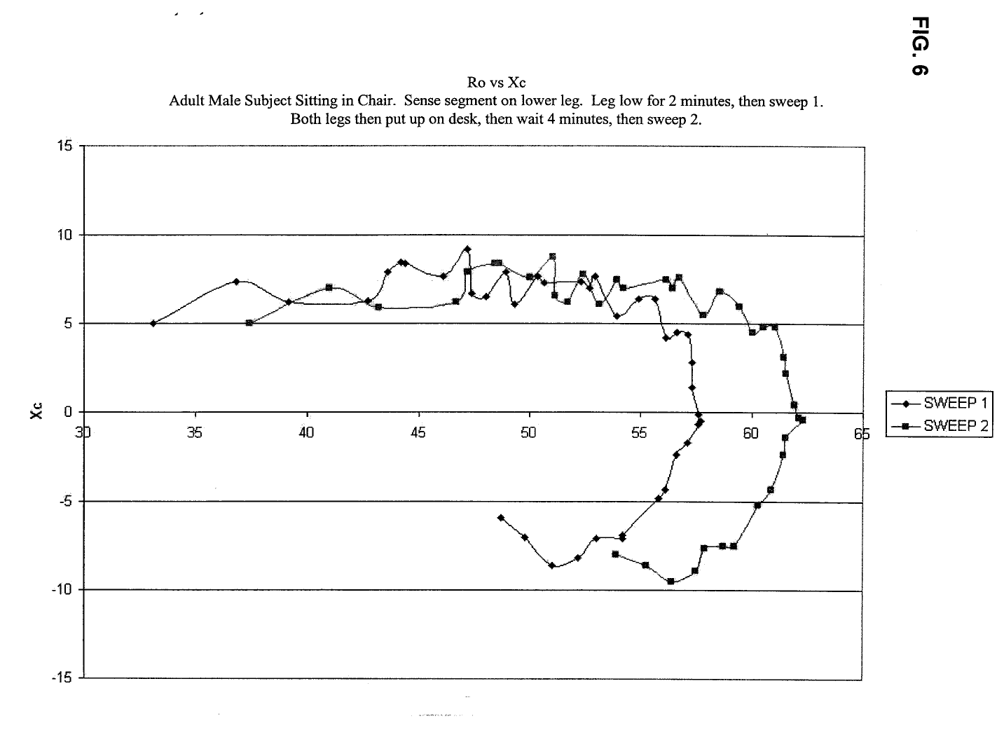

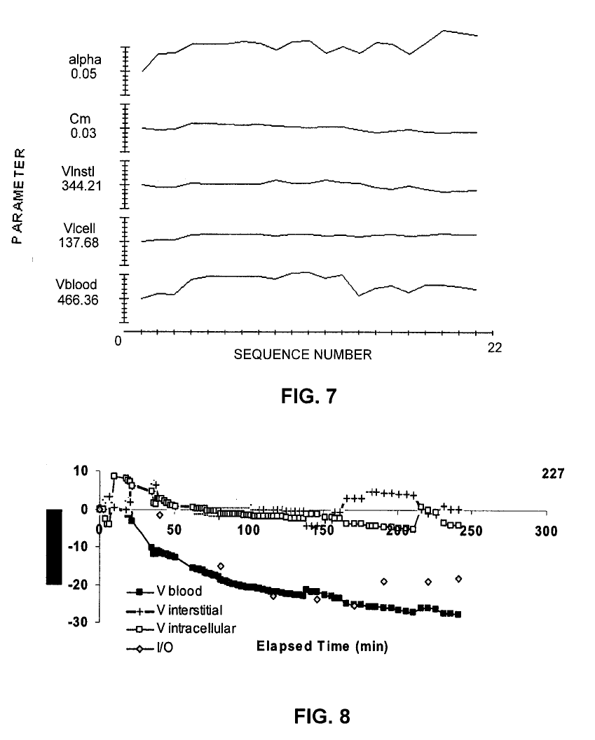

The computer 10, when executing a software product of the invention, can provide a plot of each calculated parameter and compartment volume as functions of either sweep or sequence numbers (as shown in FIG. 7). Parameter values of alpha (a locus angle), and Cm (membrane capacitance) are plotted in the top two traces. Interstitial, intracellular, and blood compartment volumes are then shown in the remaining traces as VInstl, VIcell, and Vblood respectively. The values are obtained from the resistance and reactive impedance or capacitance measurements carried out by the monitor 13. More particularly, the computer 10 is configured to store an executable deconvolution algorithm for processing the resistance and reactive impedance data to generate parameters for an R-C equivalent circuit used to generate the characterising data in the form of the interstitial, intracellular and blood compartment volumes.

Example output results from the EIS are shown in FIG. 8, demonstrating the type of measurements that can be made using electrical impedance spectroscopy. It shows changes in the intracellular and interstitial segments of the extravascular compartment in addition to changes that may take place within the vascular system over a period of four minutes. These results were obtained using a particular piece of exercise equipment and thus are only typical of a subject using this particular equipment. When using a fixed frequency version of the IPG, the capabilities of such an IPG make it possible to monitor numerous cardiovascular/fluid compartment physiologic changes that take place within various segments of the body and to investigate the interaction between body segments.

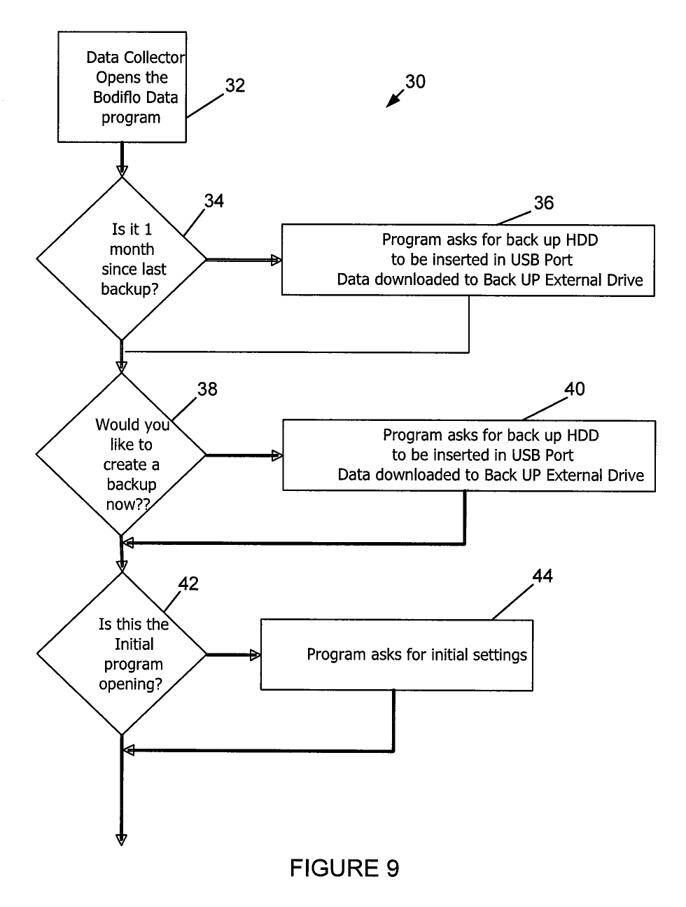

An aspect of the invention relates to the manner in which the data is collated and organised for analysis. Accordingly, the software product of the invention is configured to facilitate such collection and organisation. FIGS. 9 to 17 are flowcharts that represent a method of collecting and processing data generated by the system of the invention. In particular, the software product of the invention is configured so that when executed by at least the computer 10, at least the computer 10 carries out the steps indicated in these flowcharts. For convenience, this example is described assuming that only the computer 10 carries out the steps of the method. Thus, in the following description, the phrase “the computer 10 carries out . . . ” should be understood to mean that, when the software product of the invention is executed by at least the computer 10, at least the computer 10 carries out the relevant step. In light of that, it is to be appreciated that the steps are set out below can readily be carried out on one or more further dedicated components.

In FIG. 9, reference 30 generally indicates a flow chart representing a data collection method carried out when the software product is executed. At 32, the software product initiates a method by opening the program of the software product. At 34, the computer 10 queries a user as to the time when data was previously backed up. If the response is such that the data was backed up more than a predetermined period of time ago, the computer 10 requests that the user engage some form of removable storage device or media with the computer 10, at 36. If the response is such that the data was backed up less than said predetermined period of time ago, the computer queries as to whether or not the user would like to create a backup, at 38. If the response is positive, the computer requests that said some form of removable storage device or media be engaged with the computer 10, at 40. Otherwise, the computer 10 queries as to whether this particular opening is an initial opening at 42.

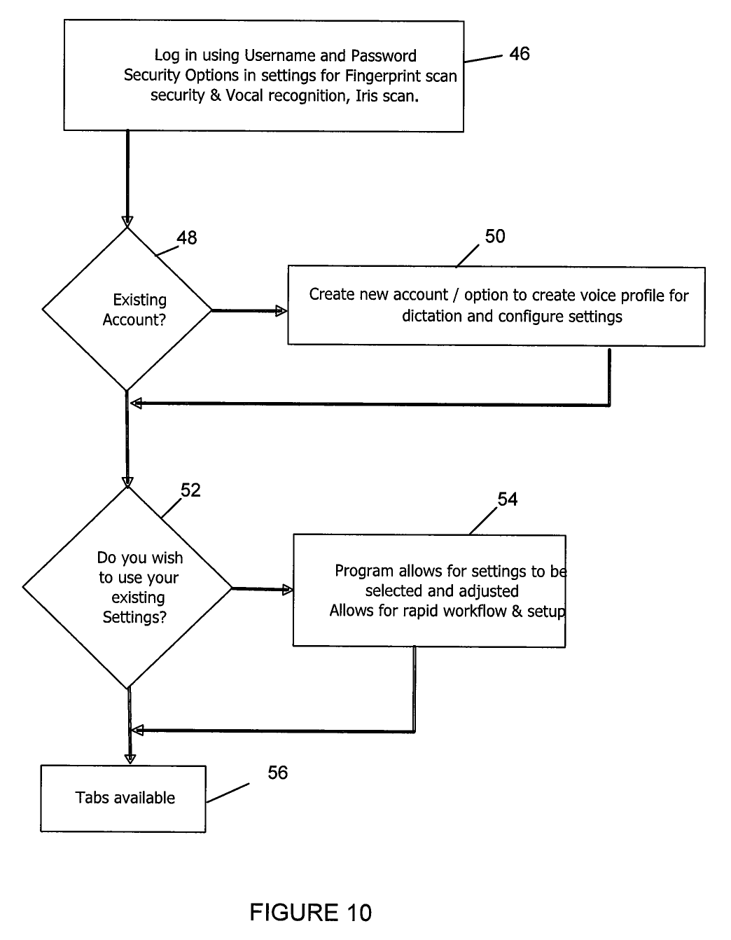

If the response is positive, the computer 10 requests the input of initial settings at 44. Otherwise, the computer 10 requests that the user log in using his or her log in details at 46 (FIG. 10). The log in details can include security options such as fingerprint data, local recognition or iris scan.

The computer 10 then queries whether or not the user is logged in to an existing account at 48. If the answer is negative, the computer 10 creates a new account and/or options to create a new voice profile for dictation and configures the settings at 50. Otherwise, the computer 10 queries whether or not the user wishes to use his or her existing settings, at 52. If the answer is positive, the computer 10 allows for settings to be selected and adjusted and also allows for workflow and set up to be carried out, at 54. Otherwise, the computer 10 makes a number of tabs available for the user at 56.

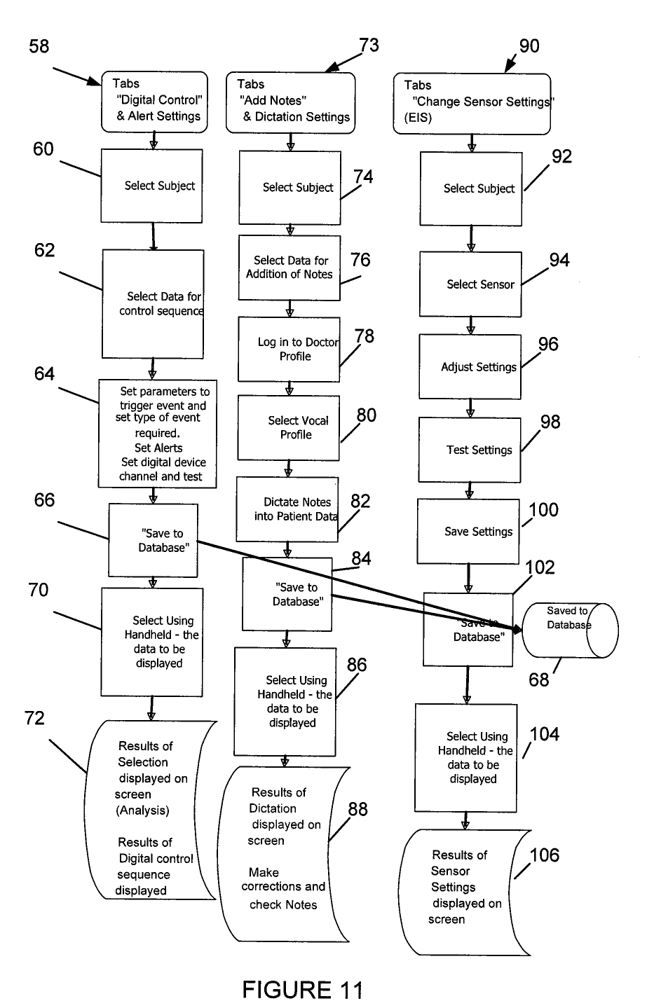

In FIG. 11, reference 58 generally indicates a flow chart for allowing a user to select digital control and alert settings. At 60, the computer 10 allows the user to select a particular subject who is being tested. At 62, the computer 10 allows the user to select data for controlling a sequence of steps. At 64, the computer 10 allows the user to set the necessary parameters to trigger events and to set the type of events required. Alerts and a digital device channel are also set and tested. At 66, the computer 10 saves data input at 60, 62, 64 to a data base 68. At 70, the computer 10 allows the user to display the data selected with a handheld device. At 72, the results of the selection are displayed on a screen for analysis and results of a digital control sequence are also displayed.

Reference 73 generally indicates a flow chart for allowing a user to add notes and to select dictation settings. At 74, the computer 10 allows the user to select a particular subject being tested. At 76, the computer 10 allows the user to select data for the addition of notes. At 78, the computer 10 allows the user to log into a particular profile of a doctor. At 80, the computer 10 allows the user to select a particular vocal profile. At 82, a computer 10 allows noted to be dictated into patient data. At 84, the computer 10 allows the user to save dictated data to the data base 68. At 86, the computer 10 allows the user to display the data selected with a handheld device. At 88, the computer displays the results of the dictation on the screen and the user is permitted to make corrections and to check notes.

Reference 90 generally indicates a flow chart for allowing a user to add notes and to change sensor settings. At 92, the computer 10 allows the user to select a particular subject being tested. At 94, the computer 10 allows the user to select a particular sensor having settings to be changed. At 96, the computer 10 allows the user to adjust those settings. At 98, the computer 10 tests the settings. At 100, the computer 10 saves the settings. The settings are saved to the data base 68 at 102. At 104, the computer 10 allows the user to display the data selected with a handheld device. At 106, the computer 10 displays the results of the sensor settings.

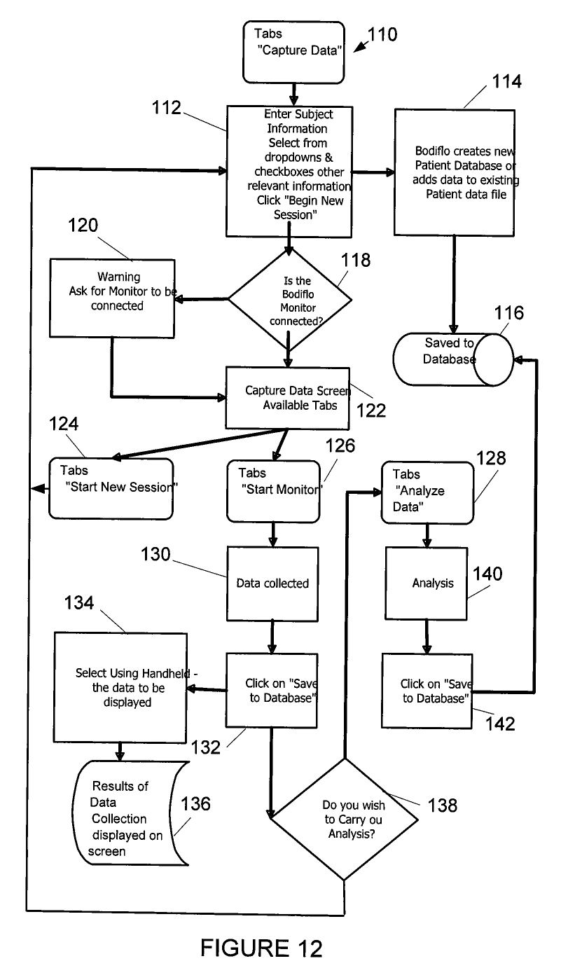

In FIG. 12, reference 110 generally indicates a flow chart for allowing a user to capture data with the computer 10 executing a software product of the invention.

At 112, the user is prompted to enter information regarding the subject. This can be done by way of drop-downs and checkboxes. At 114, the computer 10 creates a new patient data base or adds the patient data to a data base at 116. The computer 10 then queries as to whether the relevant monitor is connected at 118. If the response is negative, the computer 10 generates a warning, at 120, for connection of the monitor to the patient. If the response is positive, the computer 10 generates a number of tabs, at 122 for capturing data. More specifically, the computer 10 generates a “start new session” tab at 124, a “start monitor” tab at 126 and an “analyse data” tab at 128.

If the tab 126 is selected, the computer 10 collects the data at 130. At 132, the user is able to select a command for the computer 10 to allow the user to select data to be displayed using a handheld device at 134. At 136, the results of the data collection are displayed on a screen. If the user selects the command at 132, the computer 10 queries a user as to whether or not he or she wishes to carry out an analysis, at 138. If the response is positive, the computer 10 displays the tab 128. Then, the computer 10 performs the analysis at 140. The user is then prompted to save the results of the analysis, at 142, to the data base at 116. Otherwise, the computer 10 returns to the step at 112.

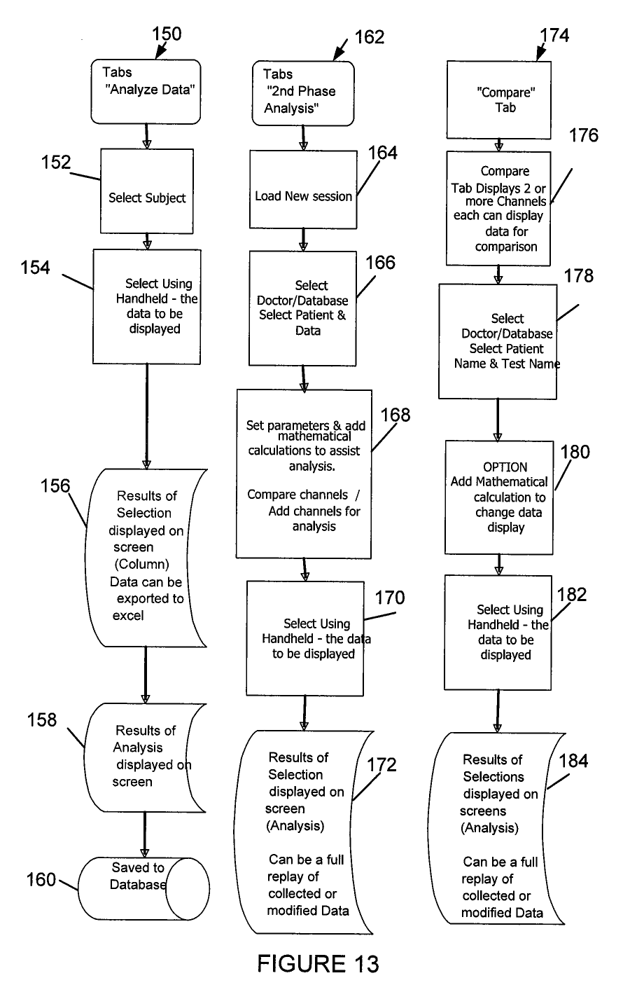

In FIG. 13, reference 150 generally indicates a flow chart representing the possible steps subsequent to the “analyse data” tab being selected. At 152, the user is prompted to select a subject. At 154, the user is prompted to select, using a handheld device, data to be displayed. At 156, the results of the selection are displayed on a screen and the computer 10 is able to export the data to a spreadsheet application. At 158, the computer 10 displays the results of the analysis on a screen. At 160, the results are saved to a data base.

Reference 162 generally indicates a flow chart representing the possible steps subsequent to a “second phase analysis” tab being selected. At 164, the computer 10 loads a new session. At 166, the computer 10 allows the user to select a doctor or a data base and also to select a patient and data relating to the patient. At 168, the computer 10 sets parameters and adds relevant mathematical calculations to assist the analysis. Also at 168, channels representing data from different regions sensed by the monitor 13 can be compared. Also, at 168, the computer 10 can add further channels for analysis. At 170, the computer 10 allows the user to select the data to be displayed with a handheld device. At 172, the computer 10 displays the results of the selection on a screen. This can be a full replay of the collected data or modified data.

Reference 174 generally indicates a flow chart representing the possible steps subsequent to a “compare” tab being selected. At 176, the computer 10 displays two or more of the channels such that the data represented by the channels can be compared. At 178, the computer 10 allows the user to select a doctor or a data base and also to select a patient and data relating to the patient. At 180, the computer 10 allows an option to add a mathematical calculation or algorithm to change the data displayed. At 182, the computer 10 allows the user to select data to be displayed with a handheld device. At 184, the computer 10 displays the results of the selection on screens. As before, this can be a full replay of the collected data or it can be modified data.

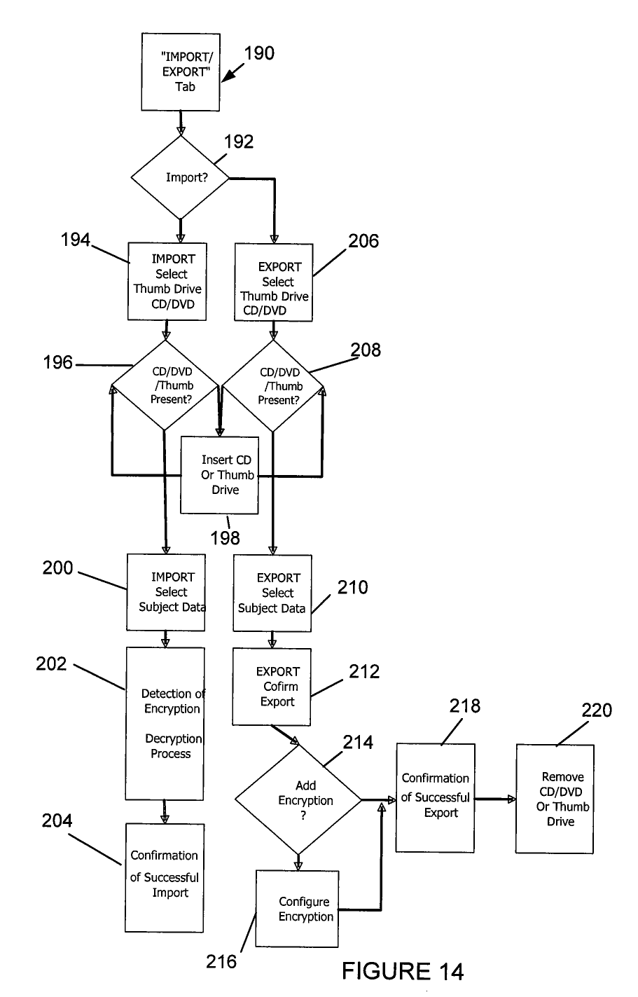

In FIG. 14, reference 190 generally indicates a flow chart representing the possible steps subsequent to an “import/export” tab being selected

At 192, the computer 10 queries whether or not data should be imported. If the response is positive, the computer 10 requests the user to select a source, such as a thumb drive, CD or DVD, at 194. At 196, the computer 10 queries as to whether or not the relevant source is present. If the answer is negative, the user is prompted to engage the relevant source with the computer 10, at 198. If the answer is positive, the computer 10 imports the relevant data, in this case data relating to the subject, at 200. At 202, the computer 10 detects whether or not encryption is present in the source and carries out a decryption process. At 204, the computer 10 confirms successful importation.

If the response to the query at 192 is negative, the computer 10 prompts the user to select a source, such as a thumb drive, CD or DVD, at 206. At 208, the computer 10 queries as to whether or not the relevant source is present. If the answer is negative, the user is prompted to engage the relevant source with the computer 10, at 198. If the answer is positive, the computer 10 exports the relevant data, in this case data relating to the subject, at 210, to the relevant source. At 212, the computer 10 confirms that the data has been exported. At 214, the computer queries whether or not encryption of the data is required. If the answer is positive, the computer 10 carries out an encryption process at 216. At 218, the computer 10 confirms successful exportation. At 220, the user is prompted to remove the relevant source.

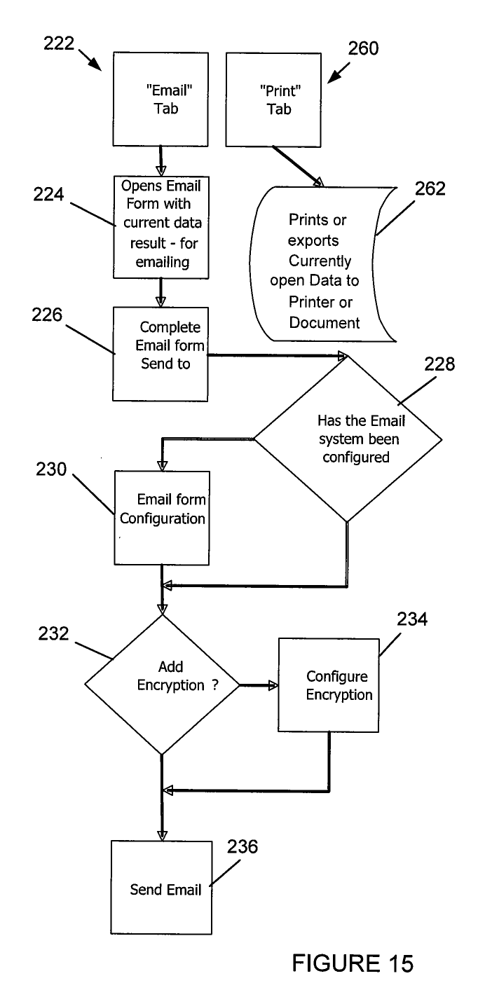

In FIG. 15, reference 222 generally indicates a flow chart representing the possible steps taken by the computer 10 when an “e-mail” tab is selected. At 224, the computer 10 opens an e-mail form in which a current data result is represented. At 226, the computer 10 prepares the e-mail form for sending, once it has been completed. At 228, the computer 10 queries as to whether or not an e-mail system has been configured. If the response is negative, the computer 10 carries out an e-mail form configuration process at 230, and queries, at 232, as to whether or not the form should be encrypted. If the response is positive, the computer 10 moves directly to the query at 232. If the response to the query at 232 is positive, the computer 10 carries out an encryption process on the form at 234 and sends the e-mail form at 236. If the response to the query is negative, the computer 10 moves directly to the step of sending the e-mail form at 236.

Reference 260 generally indicates a step taken when a “print” tab is selected. At 262, the computer 10 prince or exports currently displayed data to a printer or to a further document.

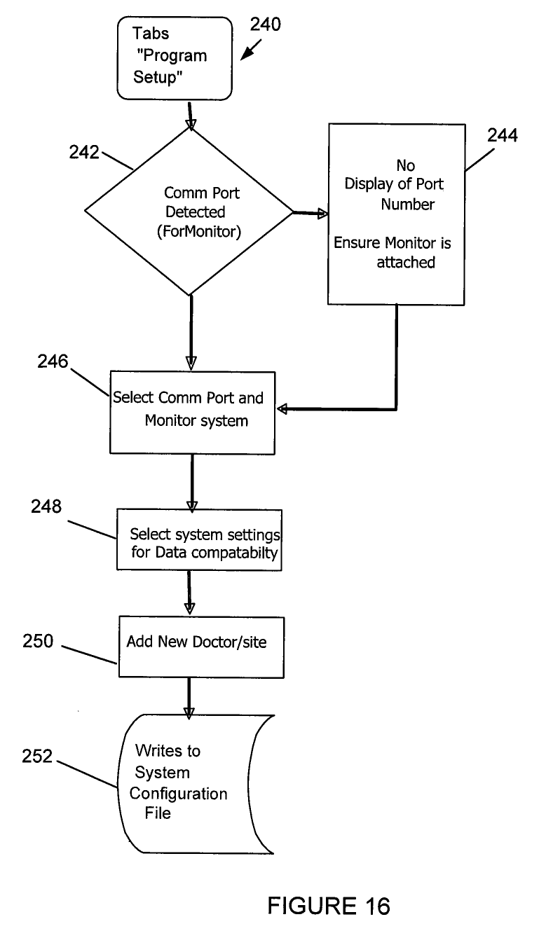

In FIG. 16, reference 240 generally indicates a flow chart representing the possible steps taken by the computer 10 when a “program setup” tab is selected. At 242, the computer 10 queries whether or not a communications port has been detected. If the response is negative, the user is prompted to ensure that the monitor is properly attached at 244. If the response is positive, the user is prompted to select the communications port and the monitor system, at 246. At 248, the computer 10 selects relevant system settings for data compatibility. At 250, the computer 10 adds doctor or site data. At 252, the computer 10 writes the setup data to a system configuration file.

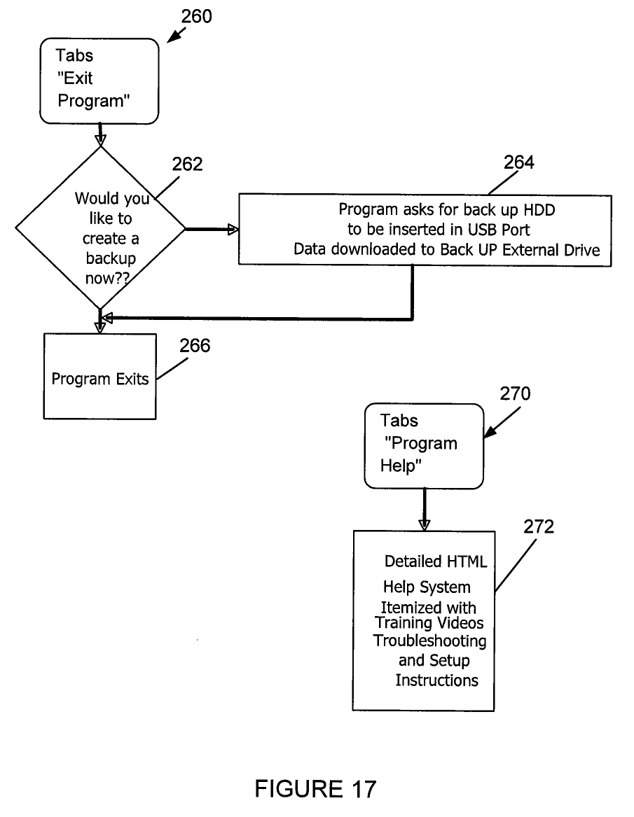

In FIG. 17, reference 260 generally indicates a flow chart representing the possible steps taken by the computer 10 when an “exit program” tab is selected. At 262, the computer 10 queries whether or not a backup of the data should be created. If the response is positive, the computer 10 prompts the user, at 264, to insert the relevant removable media to which the data to be backed up is to be written. Also at 264, the data to be backed up is written to the removable media. The computer 10 exits the program at 266. If the response is negative, the computer 10 moves directly to the step at 266.

Reference 270 shows the possible step taken when a “program help” tab is selected. The computer 10 generates a detailed HTML help system that is itemised with training videos, troubleshooting and setup instructions, at 272.

The invention may be useful in the treatment of diverse pathophysiologic fluid volume states including, for example, the management of increased intracranial pressure following trauma, the treatment of disequilibrium and hypotension during renal dialysis, the monitoring of the hydrational state of premature infants, and the investigation and diagnosis of orthostatic intolerance associated with dysautonomia.

The EIS system can be used to assess the possible compartment changes monitoring two body segments at the same time. In this way, it will provide information regarding the fluid volume redistribution between two body segments in addition to the extent of intra/extravascular fluid shifts within a single body segment. In order to achieve this, the software product of the invention can be configured to be executed by the monitor 13. Thus, the monitor 13 can be configured to cooperate with other equipment to administer treatment IS to the subject. An example of this would be a dosage meter. In one embodiment, all computer programming will be applied to a small, portable EIS that can be used to monitor the intra/extravascular compartment volumes.

By continuous measurements of segmental blood flow and fluid volume changes, it will be possible to assess all of the individual fluid compartments of the body in terms of intracellular volume, interstitial volume, and intravascular volume.

Other areas of potential application of the invention include the hydration state of premature infants and bum patients, quantification of segmental and cerebral fluid shifts that take place during orthostatic tests and exposure to microgravity, and the assessment of various countermeasures designed to reduce the stress of re-entry.

It is to be understood that the terminology employed above is for the purpose of description and should not be regarded as limiting. The foregoing embodiments are intended to be illustrative of the invention, without limiting the scope thereof. The invention is capable of being practised with various modifications and additions as will readily occur to those skilled in the art.

Accordingly, it is to be understood that the scope of the invention is not to be limited to the exact construction and operation described and illustrated, but only by the following claims which are intended, where the applicable law permits, to include all suitable modifications and equivalents within the spirit and concept of the invention.

It is envisaged that, although the invention has been described with particular reference to humans, it may also be applied to other bodies, such as animals.

Throughout this specification, including the claims, where the context permits, the term “comprise” and variants thereof such as “comprises” or “comprising” are to be interpreted as including the stated integer or integers without necessarily excluding any other integers.

1. A system for measuring physiological aspects, the system comprising:
a non-invasive monitor configured to generate monitor signals relating to fluid characteristics in the body;
a computational device operatively connected to the monitor and configured to process the monitor signals to generate characterising data relating to at least one of regional fluid volumes, intra/extracellular fluid volume ratios and blood flow; and
a data output device connected to the computational device and configured to output the characterising data.

2. A system as claimed in claim 1, in which the non-invasive monitor is one of an impedance plethysmographic (IPG) monitor and an impedance spectroscopic (EIS) monitor.

3. A system as claimed in claim 2, which includes at least two excitation electrodes for providing an electrical stimulus across selected body areas.

4. A system as claimed in claim 1, in which the monitor comprises a number of electrodes that are configured to be placed in a non-invasive manner into operative engagement with the body, such that at least one electrode engages each of a number of respective segments of the body.

5. A system as claimed in claim 1, in which the monitor is configured to use at least 20 different frequencies.

6. A system as claimed in claim 5, in which the monitor is configured to use at least 40 different frequencies at log intervals between 3 and 300 kHz.

7. A system as claimed in claim 2, in which the monitor is an EIS monitor which is configured to use a constant current transformer coupled excitation stage in conjunction with a digital demodulation stage to supply both resistant and reactive impedance components, the monitor including a microprocessor system that can be coupled to the computational device with a suitable interface.

8. A system as claimed in claim 7, in which the microprocessor system is configured to store data in the form of impedance parameters and signal waveform segments before communicating that data to the computational device, the computational device being configured to store an executable deconvolution algorithm for processing that data to generate parameters for an R-C equivalent circuit used to generate characterising data to model intravascular, interstitial and intracellular fluid spaces.

9. A system as claimed in claim 1, in which the data output device is configured to output the characterising data in one of the following forms:
a) real-time
b) a replay of previously recorded characterising data
c) together with mathematically reconstructed waveforms.

10. A method for measuring physiological characteristics, the method comprising the steps of:
engaging a non-invasive monitor with a body, the non-invasive monitor being configured to generate monitor signals relating to fluid characteristics in the body;
processing the monitor signals to generate characterising data relating to at least one of regional fluid volumes, intra/extracellular fluid volume ratios and blood flow; and
outputting the characterising data.

11. A method as claimed in claim 10, in which the step of engaging a non-invasive monitor with a body includes the step of engaging an electrode montage with the body such that five segments of the body are monitored, namely a head segment, a chest segment, a splanchnic segment, a pelvic segment and a leg segment.

12. A method as claimed in claim 11, in which the electrodes form part of a montage of ten electrodes, two electrodes being placed into engagement with each of a head segment, a chest segment, a splanchnic segment, a pelvic segment and a leg segment.

13. A method as claimed in claim 12, in which the chest segment, the splanchnic segment, the pelvic segment and the leg segment are monitored using a tetrapolar impedance system, while the head segment is monitored using a bipolar impedance configuration.

14. A method as claimed in claim 10, in which the step of processing the monitor signals includes the step of processing the monitor signals to generate resistance and reactive capacitance data and to process that data to generate the characterising data.

15. A method as claimed in claim 10, in which the step of outputting the characterising data includes the step of normalising the data and displaying the data visually.

16. A method as claimed in claim 10, in which the characterising data is output in one of the following forms:
a) real-time
b) a replay of previously recorded characterising data
c) together with mathematically reconstructed waveforms.

17. A computer program product comprising a computer useable medium including a computer readable program for measuring physiological aspects, wherein the computer readable program, when executed on a computer, causes the computer to:
process data received from a non-invasive monitor engaged with a body to generate characterising data relating to at least one of regional fluid volumes, intra/extracellular fluid volume ratios and blood flow; and
output the characterising data.

18. A computer program product as claimed in claim 17, in which the computer readable program, when executed on a computer, causes the computer to output the characterising data in one of the following forms:
a) real-time
b) a replay of previously recorded characterising data
c) together with mathematically reconstructed waveforms.
